(19) United States
(12) Patent Application Publication
(10) Pub. No.: US 2026/0041357 A1
(43) Pub. Date: Feb. 12, 2026

(54) CARDIAC MONITOR DEVICE WITH BIOCOMPATIBLE ELECTRICAL INSULATOR

(71) Applicant: Medtronic, Inc., Minneapolis, MN (US)

(72) Inventors: Blake C. Gorowsky, Lino Lakes, MN (US); Thomas J. Fick, Minneapolis, MN (US); Andrew J. Thom, Maple Grove, MN (US); Hyun J. Yoon, Vadnais Heights, MN (US); Kamal Deep Mothilal, San Diego, CA (US); Craig Wiklund, Bloomington, MN (US); Christian S. Nielsen, River Falls, WI (US); Kris A. Peterson, Edina, MN (US); Richard P. Nelson, Hudson, WI (US); Robert M. Ecker, Lino Lakes, MN (US); Jake H. Kanack, Columbia Heights, MN (US)

(21) Appl. No.: 18/998,737

(22) PCT Filed: Jul. 18, 2023
(86) PCT No.: PCT/IB2023/057320

§ 371 Date: Jan. 27, 2025

Related U.S. Application Data
(60) Provisional application No. 63/369,640, filed on Jul. 27, 2022.

(51) Int. Cl. A61B 5/29; A61B 5/00

(52) U.S. Cl. CPC A61B 5/29; A61B 5/686; A61B 5/6869; A61B 2560/0468; A61B 2562/12; A61B 2562/18

(57) ABSTRACT

An example implantable medical device includes a housing configured to house processing circuitry configured to control functioning of the implantable medical device. The housing includes an electrically conductive portion defining a cavity configured to receive the processing circuitry and a dielectric cover configured to cover the cavity and enclose the processing circuitry within the cavity. The implantable medical device further includes an electrode positioned on an outer surface of the dielectric cover and connected to the processing circuitry, the processing circuitry being further configured to monitor a physiological parameter of a patient via the electrode, and the implantable medical device further includes a biocompatible electrical insulator disposed on an outer surface of at least one of the electrically conductive portion or the dielectric cover, the biocompatible electrical insulator being configured to not be disposed on the electrode.

This application claims the benefit of U.S. Provisional Patent Application Ser. No. 63/369,640, filed 27 Jul. 2022, the entire content of which is incorporated herein by reference.

### TECHNICAL FIELD

The disclosure relates to implantable medical devices.

### BACKGROUND

Various implantable medical devices (IMDs) have been clinically implanted or proposed for therapeutically treating or monitoring one or more conditions of a patient. Such devices may be adapted to monitor or treat conditions or functions relating to heart, muscle, nerve, brain, stomach, endocrine organs or other organs and their related functions. Advances in design and manufacture of miniaturized electronic and sensing devices have enabled development of implantable devices capable of therapeutic as well as diagnostic functions such as pacemakers, cardioverters, defibrillators, biochemical sensors, implantable loop recorders, and pressure sensors, among others. Such devices may be associated with leads that position electrodes or sensors at a desired location or may be leadless with electrodes integrated into the device housing. These devices may have the ability to wirelessly transmit data either to another device implanted in the patient or to another instrument located externally of the patient, or both.

Although implantation of some devices requires a surgical procedure (e.g., pacemakers, defibrillators, etc.), other devices may be small enough to be delivered and placed at an intended implant location in a relatively noninvasive manner, such as by a percutaneous delivery catheter, transvenously, or using a subcutaneous delivery tool. As one example, subcutaneously implantable monitors have been proposed and used to monitor heart rate and rhythm, as well as other physiological parameters, such as patient posture and activity level. Such direct in vivo measurement of physiological parameters may provide significant information for clinicians to facilitate diagnostic and therapeutic decisions.

### SUMMARY

The disclosure describes implantable medical devices (IMDs) including a biocompatible electrical insulator to electrically isolate a sensing electrode and an antenna of the IMD, and associated techniques for manufacturing IMDs including a biocompatible electrical insulator. An IMD comprises a housing defining a cavity and configured to house processing circuitry configured to control the functioning of the IMD. The housing comprises an electrically conductive portion defining the cavity, e.g., a titanium shell, and a dielectric cover, e.g., a sapphire cover, configured to enclose the processing circuitry within the cavity. The IMD further comprises electrodes (e.g., one or more electrodes), positioned on an outer surface of the dielectric cover and connected to the processing circuitry, wherein the processing circuitry is configured to monitor a physiological parameter of a patient via the electrode. The IMD further comprises an antenna, at least a portion of which is positioned on the surface of the dielectric cover and is configured to send and receive information via electromagnetic radiation (e.g., wireless communication radio waves, such as according to the Bluetooth® protocol).

The electrodes are configured to be in contact with tissue and/or fluids of the patient in order to monitor the physiological parameter of a patient. In some examples, the electrodes comprise an anode and a cathode configured to be in contact with tissue and/or fluids of the patient and separated by a particular distance. If in electrical contact with tissue and/or fluids of the patient, the electrically conductive portion of the housing, while not in electrical contact with the electrodes, are in relatively close proximity to the electrodes, and may provide an electrical conduction path having a reduced electrical resistance (relative to patient tissue and/or fluids) between the electrodes. This condition could effectively “short” the electrodes and cause erroneous and/or missed measurements. For example, the electrically conductive portion of the housing may effectively be a conductor between tissue and/or fluids at the positions of the electrodes and cause the tissue and/or fluids at those positions to be at the same electrical potential and/or voltage when they otherwise would not be, and which may “block” biopotentials from being sensed by the electrodes.

Additionally, if the portion of the antenna of the IMD on the outer surface of the dielectric cover is in contact with surrounding tissue and/or fluids of the patient, the conductivity of the surrounding tissue and/or fluids may change and/or reduce the electrical current in the antenna caused by the communication radio waves and degrade the communication signal.

As described herein, an IMD comprises a biocompatible electrical insulator, e.g., parylene, disposed on an outer surface of at least one of the electrically conductive portion or the dielectric cover and configured to electrically isolate at least one of the antenna or a portion of the housing from electrical contact with surrounding tissue and/or fluids. As described herein, a method of disposing a biocompatible electrical insulator on at least a portion of the electrically conductive portion of the housing and/or a portion of the dielectric cover includes masking the electrodes, coating at least a portion of the electrically conductive portion of the housing and/or a portion of the dielectric cover with the biocompatible electrical insulator, and removing the mask without delaminating the coated biocompatible electrical insulator from the surface of the housing.

The devices, systems, and techniques of this disclosure may provide one or more advantages and benefits. For instance, an IMD comprising a biocompatible electrical insulator may provide improved communications (speed, reliability, bandwidth, range, or the like), via electrically isolating at least a portion of the antenna from tissue thereby improving the amount of electrical current in the antenna (e.g., reducing “current leak” from the antenna) and/or reducing antenna noise. Additionally, an IMD comprising a biocompatible electrical insulator may provide improved sensitivity and reliability of sensing a physiological parameter via electrically isolating at least a portion of the conductive housing from tissue and/or fluids and thereby reducing and/or preventing “shorting” of the electrodes and/or “blocking” biopotentials from being sensed by the electrodes.

In one example, this disclosure describes an implantable medical device including: a housing configured to house processing circuitry, wherein the processing circuitry is configured to control functioning of the implantable medical device, wherein the housing comprises: an electrically conductive portion defining a cavity configured to receive the processing circuitry; and a dielectric cover configured to cover the cavity and enclose the processing circuitry within the cavity; an electrode positioned on an outer surface of the dielectric cover and connected to the processing circuitry, wherein the processing circuitry is configured to monitor a physiological parameter of a patient via the electrode; and a biocompatible electrical insulator disposed on an outer surface of at least one of the electrically conductive portion or the dielectric cover, wherein the biocompatible electrical insulator is configured to not be disposed on the electrode.

In another example, this disclosure describes a method including: masking an electrode of an implantable medical device with a mask includes a housing configured to house processing circuitry, wherein the processing circuitry is configured to control functioning of the implantable medical device, wherein the housing comprises: an electrically conductive portion defining a cavity configured to receive the processing circuitry; and a dielectric cover configured to cover the cavity and enclose the processing circuitry within the cavity; and the electrode positioned on an outer surface of the dielectric cover and connected to the processing circuitry, wherein the processing circuitry is configured to monitor a physiological parameter of a patient via the electrode; coating at least a portion of an outer surface of the housing and an outer surface of the mask with a biocompatible electrical insulator; removing the mask without delaminating the coated biocompatible electrical insulator from the surface of the housing.

This summary is intended to provide an overview of the subject matter described in this disclosure. It is not intended to provide an exclusive or exhaustive explanation of the apparatus and methods described in detail within the accompanying drawings and description below. The details of one or more aspects of the disclosure are set forth in the accompanying drawings and the description below. Other features, objects, and advantages will be apparent from the description, drawings, and claims.

### BRIEF DESCRIPTION OF THE FIGURES

The details of one or more examples of this disclosure are set forth in the accompanying drawings and the description below. Other features, objects, and advantages of this disclosure will be apparent from the description, drawings, and claims.

FIG. 1 is a conceptual drawing illustrating an example medical device system in conjunction with a patient, according to various examples described in this disclosure.

FIG. 2A is a conceptual side-view diagram illustrating an example configuration of the implantable medical device (IMD) and biocompatible electrical insulator of the medical system of FIG. 1, according to various examples described in this disclosure.

FIG. 2B is a conceptual perspective view diagram illustrating the example configuration of the implantable medical device (IMD) and biocompatible electrical insulator of FIG. 2A, according to various examples described in this disclosure.

FIG. 3 is a functional block diagram illustrating an example configuration of the implantable medical device (IMD) of the medical system of FIG. 1, according to various examples described in this disclosure.

FIG. 4 is a perspective view of an example IMD positioned within a mount including coating masks, according to various examples described in this disclosure.

FIG. 5 is a perspective cross-sectional view of a portion of the example IMD positioned within the example mount of FIG. 4, according to various examples described in this disclosure.

FIG. 6 is an enlarged perspective cross-sectional view of a portion of the example IMD and coating mask of FIG. 4, according to various examples described in this disclosure.

FIG. 7 is a further enlarged perspective cross-sectional view of the portion of the example IMD and coating mask indicated in FIG. 6 including a coated biocompatible electrical insulator, according to various examples described in this disclosure.

FIG. 8 is a perspective cross-sectional view of an example biocompatible electrical insulator coated onto the example IMD of FIG. 7, according to various examples described in this disclosure.

FIG. 9 is front elevation view of an example IMD including an example biocompatible electrical insulator 16, according to various examples described in this disclosure.

FIG. 10 is a flow diagram of an example method of manufacturing an implantable medical device including a biocompatible electrical insulator, according to various examples described in this disclosure.

In the figures, use of a same reference number or a same reference number with a letter extension may be used to indicate a same or corresponding device or element when used in a same drawing or in different drawings. In addition, unless otherwise indicated, devices and/or other objects such as a patient, an implantable medical device, or an electrical device such as an electrical coil, are not necessarily illustrated to scale relative to each other and/or relative to an actual example of the item being illustrated. In particular, various drawings provided with this disclosure illustrate a “patient” represented by a human-shaped outline and are not to be considered drawn to scale relative to an actual human patient or with respect to other objects illustrated in the same figure unless otherwise specifically indicated in the figure for example by dimensional indicators, or for example as otherwise described in the text of the disclosure.

### DETAILED DESCRIPTION

A variety of types of medical devices sense cardiac electrograms (EGMs) and/or other physiological signals or parameters of a patient. Some medical devices that sense cardiac EGMs and/or other patient signals or parameters are non-invasive, e.g., using a plurality of electrodes placed in contact with external portions of the patient, such as at various locations on the skin of the patient to sense cardiac EGMs. The electrodes used to monitor the cardiac EGM in these non-invasive processes may be attached to the patient using an adhesive, strap, belt, or vest, as examples, and electrically coupled to a monitoring device, such as an electrocardiogramalter monitor, or other electronic device. The electrodes are configured to sense electrical signals associated with the electrical activity of the heart or other cardiac tissue of the patient, and to provide these sensed electrical signals to the electronic device for further processing and/or display of the electrical signals. The non-invasive devices and methods may be utilized on a temporary basis, for example to monitor a patient during a clinical visit, such as during a doctor's appointment, or for example for a predetermined period of time, for example for one day (twenty-four hours), or for a period of several days.

External devices that may be used to non-invasively sense and monitor cardiac EGMs include wearable devices with electrodes configured to contact the skin of the patient, such as patches, watches, or necklaces. One example of a wearable physiological monitor configured to sense a cardiac EGM is the SEEQ™ Mobile Cardiac Telemetry System, available from Medtronic plc, of Dublin, Ireland. Such external devices may facilitate relatively longer-term monitoring of patients during normal daily activities and may periodically transmit collected data to a network service, such as the Medtronic Carelink™ Network.

Some implantable medical devices (IMDs) also sense and monitor cardiac EGMs. The electrodes used by IMDs to sense cardiac EGMs are typically integrated with a housing of the IMD and/or coupled to the IMD via one or more elongated leads. Example IMDs that monitor cardiac EGMs include pacemakers and implantable cardioverter-defibrillators, which may be coupled to intravascular or extravascular leads, as well as pacemakers with housings configured for implantation within the heart, which may be leadless. An example of pacemaker configured for intracardiac implantation is the Micra™ Transcatheter Pacing System, available from Medtronic plc. Some IMDs that do not provide therapy, e.g., implantable patient monitors, sense cardiac EGMs. Examples of such IMDs are the Reveal LINQ™ and LINQ II™ Insertable Cardiac Monitor (ICMs), available from Medtronic plc, which may be inserted subcutaneously. Such IMDs may facilitate relatively longer-term monitoring of patients during normal daily activities and may periodically transmit collected data to a network service, such as the Medtronic Carelink™ Network.

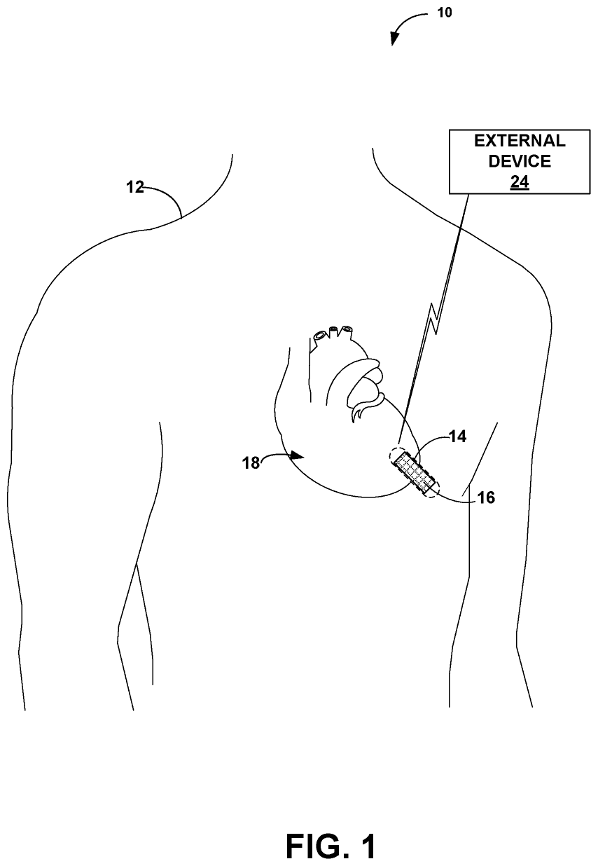

FIG. 1 is a conceptual drawing illustrating an example medical system 10 in conjunction with a patient 12 according to various examples described in this disclosure. The systems, devices, and methods described in this disclosure may include examples configurations of a biocompatible electrical insulator 16 disposed on an IMD 14, as illustrated and described with respect to FIG. 1. For purposes of this description, knowledge of cardiovascular anatomy and functionality is presumed, and details are omitted except to the extent necessary or desirable to explain the context of the techniques of this disclosure. System 10 includes IMD 14 having biocompatible electrical insulator 16, implanted at or near the site of a heart 18 of a patient 12 and an external computing device 24. The systems, devices, and methods described herein may provide infection control and migration control of IMD 14.

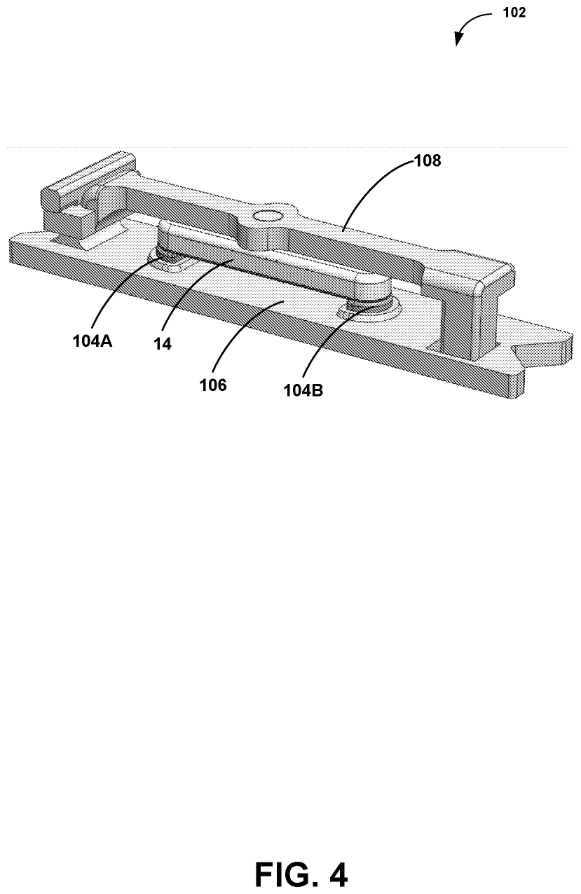

The example techniques may be used with IMD 14, which may be in wireless communication with at least one of external device 24 and other devices not pictured in FIG. 1. In some examples, IMD 14 is implanted outside of a thoracic cavity of patient 12 (e.g., subcutaneously in the pectoral location illustrated in FIG. 1). IMD 14 may be positioned near the sternum near or just below the level of the heart of patient 12, e.g., at least partially within the cardiac silhouette. IMD 14 includes a plurality of electrodes 48 (FIG. 5) and is configured to sense a cardiac electrogram (EGM) via the plurality of electrodes. In some examples, IMD 14 takes the form of the LINQ™ or LINQ II™ ICM, or another ICM similar to, e.g., a version or modification of, the LINQ™ or LINQ II™ ICM. Although described primarily in the context of examples in which IMD 14 is an ICM, in various examples, IMD 14 may represent a cardiac monitor, a defibrillator, a cardiac resynchronization pacer/defibrillator, a pacemaker, an implantable pressure sensor, a neurostimulator, or any other implantable or external medical device.

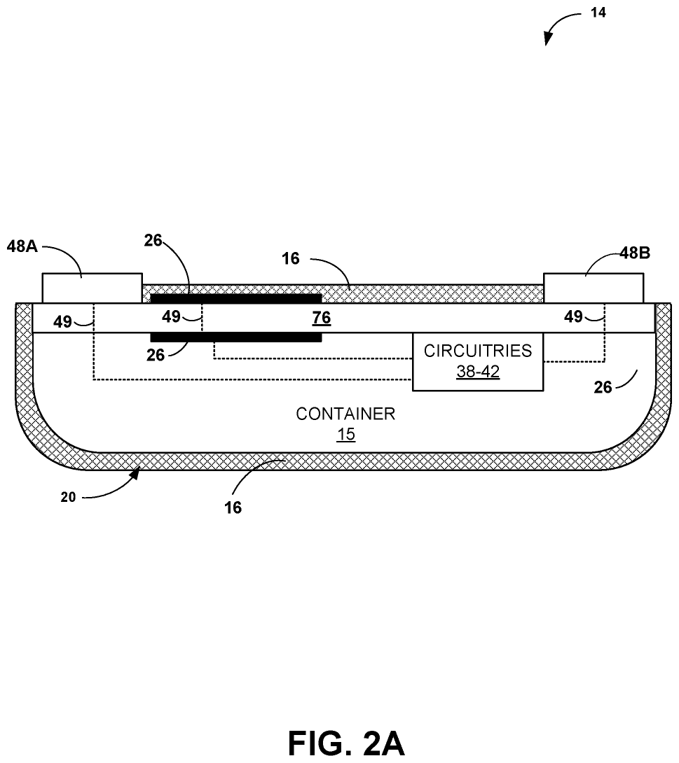

In some examples, IMD 14 is defined by a length L, a width W and thickness or depth D and is in the form of an elongated rectangular prism wherein the length L is much larger than the width W, which in turn is larger than the depth D, as illustrated in FIG. 2B below. In one example, the geometry of the IMD 14—in particular a width W greater than the depth D—is selected to allow IMD 14 to be inserted under the skin of the patient using a minimally invasive procedure and to remain in the desired orientation during insert. For example, IMD 14 may include a radial asymmetry (notably, a rectangular shape) along the longitudinal axis that maintains the device in the proper orientation following insertion. For example, in one example the spacing between electrode 48A and electrode 48B may range from 30 millimeters (mm) to 55 mm, 35 mm to 55 mm, and from 40 mm to 55 mm and may be any range or individual spacing from 25 mm to 60 mm. In another example the spacing between electrode 48A and electrode 48B may range from 15 mm to 30 mm, 17 mm to 28 mm, and from 20 mm to 28 mm and may be any range or individual spacing from 12 mm to 30 mm. In addition, IMD 14 may have a length L that ranges from 30 mm to about 70 mm. In other embodiments, the length L may range from 40 mm to 60 mm, 45 mm to 60 mm and may be any length or range of lengths between about 30 mm and about 70 mm. In some examples, IMD 14 may have a length L that ranges from 15 mm to about 35 mm, or from 20 mm to 30 mm, 22 mm to 30 mm and may be any length or range of lengths between about 15 mm and about 35 mm. In addition, the width W of a major surface of IMD 14, e.g., insulative cover 76 in the example shown, may range from 3 mm to 10 mm and may be any single or range of widths between 3 mm and 10 mm, or may range from 1.5 mm to 5 mm and may be any single or range of width between 1.5 mm and 5 mm. The thickness of depth D of IMD 14 may range from 2 mm to 9 mm, or from 1.5 mm to 4.5 mm. In other embodiments, the depth D of IMD 14 may range from 2 mm to 5 mm and may be any single or range of depths from 2 mm to 9 mm, or may range from 1 mm to 2.5 mm and may be any single or range of depts from 1 mm to 4.5 mm. In addition, IMD 14 according to an example of the present invention has a geometry and size designed for ease of implant and patient comfort. Examples of IMD 14 described in this disclosure may have a volume of 3 cubic centimeters (cm) or less, 1.5 cubic cm or less or any volume between 3 and 1.5 cubic centimeters, or may have a volume of 1.5 cubic centimeters (cm) or less, 0.75 cubic cm or less or any volume between 1.5 and 0.75 cubic centimeters.

External device 24 may be a computing device with a display viewable by the user and an interface for providing input to external device 24 (i.e., a user input mechanism). In some examples, external device 24 may be a notebook computer, tablet computer, workstation, one or more servers, cellular phone, personal digital assistant, or another computing device that may run an application that enables the computing device to interact with IMD 14. External device 24 is configured to communicate with IMD 14 and, optionally, another computing device (not illustrated in FIG. 1), via wireless communication. External device 24, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies).

External device 24 may be used to configure operational parameters for IMD 14. External device 24 may be used to retrieve data from IMD 14. The retrieved data may include values of physiological parameters measured by IMD 14, indications of episodes of arrhythmia or other maladies detected by IMD 14, and physiological signals recorded by IMD 14. For example, external device 24 may retrieve cardiac EGM segments recorded by IMD 14, e.g., due to IMD 14 determining that an episode of arrhythmia or another malady occurred during the segment, or in response to a request to record the segment from patient 12 or another user. In some examples, one or more remote computing devices may interact with IMD 14 in a manner similar to external device 24, e.g., to program IMD 14 and/or retrieve data from IMD 14, via a network.

In various examples, IMD 14 may include one or more additional sensor circuits configured to sense a particular physiological or neurological parameter associated with patient 12, or may comprise a plurality of sensor circuits, which may be located at various and/or different positions relative to patient 12 and/or relative to each other and may be configured to sense one or more physiological parameters associated with patient 12.

For example, IMD 14 may include a sensor operable to sense a body temperature of patient 12 in a location of the IMD 14, or at the location of the patient where a temperature sensor coupled by a lead to IMD 14 is located. In another example, IMD 14 may include a sensor configured to sense motion, such as steps taken by patient 12 and/or a position or a change of posture of patient 12. In various examples, IMD 14 may include a sensor that is configured to detect breaths taken by patient 12. In various examples, IMD 14 may include a sensor configured to detect heartbeats of patient 12. In various examples, IMD 14 may include a sensor that is configured to measure systemic blood pressure of patient 12.

In some examples, one or more of the sensors comprising IMD 14 may be implanted within patient 12, that is, implanted below at least the skin level of the patient. In some examples, one or more of the sensors of IMD 14 may be located externally to patient 12, for example as part of a cuff or as a wearable device, such as a device imbedded in clothing that is worn by patient 12. In various examples, IMD 14 may be configured to sense one or more physiological parameters associated with patient 12, and to transmit data corresponding to the sensed physiological parameter or parameters to the external device 24, as represented by the lightning bolt coupling IMD 14 to the external device 24.

Transmission of data from IMD 14 to external device 24 in various examples may be performed via wireless transmission, using for example any of the formats for wireless communication described above. In various examples, IMD 14 may communicate wirelessly to an external device (e.g., an instrument or instruments) other than or in addition to external device 24, such as a transceiver or an access point that provides a wireless communication link between IMD 14 and a network. Examples of communication techniques used by any of the devices described above with respect to FIG. 1 may include radiofrequency (RF) telemetry, which may be an RF link established via Bluetooth®, Wi-Fi, or medical implant communication service (MICS).

In some examples, system 10 may include more or fewer components than depicted in FIG. 1. For example, in some examples, system 10 may include multiple additional IMDs, such as implantable pacemaker devices or other IMDs, implanted within patient 12. In these examples, IMD 14 may function as a hub device for the other IMDs. For example, the additional IMDs may be configured to communicate with the IMD 14, which would then communicate to the external device 24, such as a user's smartphone, via a low-energy telemetry protocol. IMD 14 may provide a theoretically infinite energy capacity, in that IMD 14 may not need to be replaced or otherwise removed. Accordingly, IMD 14 may provide the ability to more-frequently telemeter information, as well as more-active titration of therapies.

For the remainder of the disclosure, a general reference to a medical device system may refer collectively to include any examples of medical device system 10, a general reference to IMD 14 may refer collectively to include any examples of IMD 14, a general reference to sensor circuits may refer collectively to include any examples of sensor circuits of IMD 14, and a general reference to an external device may refer collectively to any examples of external device 24.

FIG. 2A is a conceptual side-view diagram illustrating an example configuration of the IMD 14 and biocompatible electrical insulator 16 of medical system 10 of FIG. 1, and FIG. 2B is a conceptual perspective view diagram illustrating the example configuration of the IMD 14 and biocompatible electrical insulator 16 of FIG. 2A, according to various examples described in this disclosure.

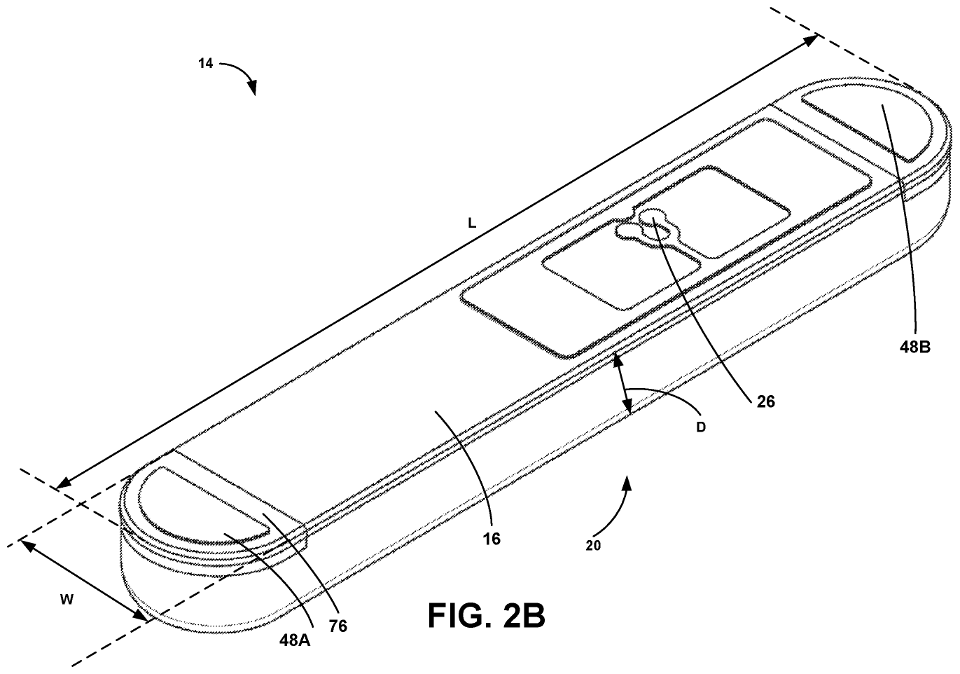

In the examples shown in FIGS. 2A-2B, IMD 10 may include a leadless, subcutaneously implantable monitoring device having a container 15 and an insulative cover 76. Electrode 48A and electrode 48B (collectively “electrodes 48”) may be formed or placed on an outer surface of cover 76. Circuitries 36-42, described below with respect to FIG. 3, may be formed or placed on an inner surface of cover 76, or within container 15. In some examples, antenna 26 is formed or placed on the inner surface of cover 76. In other examples, antenna 26 is formed or placed on the outer surface of cover 76, and in other examples, antenna 26 may be formed or placed at least partially on the inner surface and partially on the outer surface of cover 76. In some examples, insulative cover 76 may be positioned over an open container 15 such that container 15 and cover 76 form housing 20 and enclose circuitries 36-42 (and in some cases antenna 26) and protect the circuitries from fluids such as body fluids. One or more of circuitries 36-42 may be formed on the inner side of insulative cover 76, such as by using flip-chip technology. Insulative cover 76 may be flipped onto a container 15. When flipped and placed onto container 15, the components of IMD 10 formed on the inner side of insulative cover 76 may be positioned in a gap defined by container 15. Electrodes 48 and antenna 26 (when placed or formed on the outer surface of cover 76) may be electrically connected to sensing circuitry 42 and communication circuitry 38 (illustrated in FIG. 3), respectively, through one or more vias 49 formed through insulative cover 76. Insulative cover 76 may be formed of sapphire (i.e., corundum), glass, and/or any other suitable insulating material. Container 15 may be formed from any suitable material configured to house circuitries 36-42, support and mate with cover 26 to isolate circuitries 36-42 for contact with tissue and/or fluids of patient 12, and to be implantable within patient 12. In some examples, container 15 may house a battery or other power source. In some examples, container 15 may also be electrically conductive. For example, container 15 may be formed from titanium or any other suitable material (e.g., a biocompatible material). Electrodes 48 may be formed from any of stainless steel, titanium, platinum, iridium, or alloys thereof. In addition, electrodes 48 may be coated with a material such as titanium nitride or fractal titanium nitride, although other suitable materials and coatings for such electrodes may be used.

Biocompatible electrical insulator 16 is disposed on housing 20. In the example shown, biocompatible electrical insulator 16 is disposed on insulative cover 76 and the outer surface of container 15. In some examples, biocompatible electrical insulator 16 may be disposed on all or a portion of any outer surface of IMD 14 and/or housing 20, except for outer surfaces of electrodes 48. For example, biocompatible electrical insulator 16 may be disposed on all or a portion of container 15, insulative cover 76, or at least a portion of both container 15 and insulative cover 76. In some examples, biocompatible electrical insulator 16 may be disposed on greater than or equal to 20% of the surface area of housing 20, on greater than or equal to 50% of the surface area of housing 20, on greater than or equal to 75% of the surface area of housing 20, on greater than or equal to 90% of the surface area of housing 20, or any suitable surface area of housing 20. In some examples, biocompatible electrical insulator 16 may be disposed on substantially all of housing 20 surface area that is not a sensor electrode, e.g., electrode 48A or 48B. In some examples, biocompatible electrical insulator 16 may be disposed on greater than 55% of housing 20 surface area that is not electrode 48A or 48B and in some other examples, biocompatible electrical insulator 16 may be disposed on greater than 90% of housing 20 surface area that is not electrode 48A or 48B.

In some examples, biocompatible electrical insulator 16 may be disposed on at least a portion of housing 20 surface area by vacuum depositing a coating of biocompatible electrical insulator 16 on housing 20. For example, biocompatible electrical insulator 16 may conform to the shape of housing 20 and be configured to adhere and/or attach to the outer surface of housing 20 and/or outer surfaces of components on the outer surface of housing 20, e.g., antenna 26, excluding electrodes 48. In some examples, biocompatible electrical insulator 16 may be disposed on a portion of the outer surface of cover 76 to as to cover and/or encapsulate antenna 26, or at least a portion of antenna 26 disposed on the outer surface of cover 76, and biocompatible electrical insulator 16 may be alternatively or additionally disposed on all of the outer surface of container 16, e.g., to cover and/or encapsulate container 15 so as to electrically isolate container 15 from electrical contact with tissue and/or fluid of patient 12.

Biocompatible electrical insulator 16 may be configured to not interfere with the efficacy of IMD 14, e.g., electrodes 48, receiving a physiological signal. In the example shown, biocompatible electrical insulator 16 is disposed on surface areas of insulative cover 76 and container 15 not including electrodes 48. In some examples, biocompatible electrical insulator 16 is configured to improve the efficacy of IMD 14. For example, biocompatible electrical insulator 16 may be configured to improve antenna 26 receiving and/or sending communication signals by encapsulating and electrically isolating at least a portion of antenna 26 disposed on an outer surface of cover 76 from tissue and/or fluids of patient 12.

In some examples, biocompatible electrical insulator 16 may be disposed on housing 20 in a pattern. For example, biocompatible electrical insulator 16 may be disposed on a first area of housing 20 and not disposed on a second area of housing 20. In some examples, biocompatible electrical insulator 16 may be etched to form the pattern. For example, biocompatible electrical insulator 16 may be disposed on housing 20 and may be removed and/or etched away at one or more areas of housing 20, e.g., electrode 48 areas and/or areas opposite and/or including antenna 26. In other examples, biocompatible electrical insulator 16 may be patterned via etching or any suitable method, and then disposed on housing 20. For example, the pattern of patterned biocompatible electrical insulator 16 may align with features of IMD 14, e.g., etched away and/or removed portions of biocompatible electrical insulator 16 may align with electrodes 48 or any other suitable feature.

In some examples, biocompatible electrical insulator 16 may be disposed with a thickness, e.g., at least 25 micrometers thick, at least 100 micrometers thick, at least 1 millimeter thick, at least 5 millimeters thick, at least 10 millimeters thick, or any suitable thickness. In some examples, biocompatible electrical insulator 16 may be disposed on housing 20 having a substantially uniform layer thickness. In other examples, biocompatible electrical insulator 16 may be disposed on housing 20 having a layer thickness that varies, e.g., biocompatible electrical insulator 16 may be disposed on container 15 with a thicker layer thickness than biocompatible electrical insulator 16 disposed on cover 76, or vice versa. In some examples, biocompatible electrical insulator 16 may comprise parylene.

FIG. 3 is a functional block diagram illustrating an example configuration of the implantable medical device (IMD) and of the medical system of FIG. 1. In the illustrated example, IMD 14 includes processing circuitry 40, memory 36, communication circuitry 38, communication antenna 26, sensing circuitry 42, sensor(s) 44, accelerometer(s) 46, and electrodes 48A and 48B (collectively, “electrodes 48”).

Although the illustrated example includes two electrodes 48, IMDs including or coupled to one electrode 48, or more than two electrodes 48, may implement the techniques of this disclosure in some examples.

Processing circuitry 40 may include fixed function circuitry and/or programmable processing circuitry. Processing circuitry 40 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 40 may include multiple components, such as any combination of one or more microprocessors, one or more controllers, one or more DSPs, one or more ASICs, or one or more FPGAs, as well as other discrete or integrated logic circuitry. The functions attributed to processing circuitry 40 herein may be embodied as software, firmware, hardware or any combination thereof.

Sensing circuitry 42 is coupled to electrodes 48 and is configured to monitor one or more physiological parameters of a patient. Sensing circuitry 42 may sense signals from electrodes 48, e.g., to produce a cardiac EGM, in order to facilitate monitoring the electrical activity of the heart. Sensing of a cardiac EGM may be done to determine heart rates or heart rate variability, or to detect arrhythmias (e.g., tachyarrhythmias or bradycardia). Sensing circuitry 42 may additionally monitor impedance or other electrical phenomena via electrodes 48. Sensing circuitry 42 also may monitor signals from sensors 44, which may include one or more accelerometers 46, pressure sensors, and/or optical sensors, as examples. In some examples, sensing circuitry 42 may include one or more filters and amplifiers for filtering and amplifying signals received from electrodes 48 and/or sensors 44. In some examples, sensing circuitry 42 may sense or detect physiological parameters, such as heart rate, blood pressure, respiration, and other physiological parameters associated with a patient.

Sensing circuitry 42 and/or processing circuitry 40 may be configured to detect cardiac depolarizations (e.g., P-waves of atrial depolarizations or R-waves of ventricular depolarizations) when the cardiac EGM amplitude crosses a sensing threshold. For cardiac depolarization detection, sensing circuitry 42 may include a rectifier, filter, amplifier, comparator, and/or analog-to-digital converter, in some examples. In some examples, sensing circuitry 42 may output an indication to processing circuitry 40 in response to sensing of a cardiac depolarization. In this manner, processing circuitry 40 may receive detected cardiac depolarization indicators corresponding to the occurrence of detected R-waves and P-waves in the respective chambers of heart. Processing circuitry 40 may use the indications of detected R-waves and P-waves for determining inter-depolarization intervals, heart rate, and detecting arrhythmias, such as tachyarrhythmias, bradyarrhythmias, and asystole.

Sensing circuitry 42 may also provide one or more digitized cardiac EGM signals to processing circuitry 40 for analysis, e.g., for use in cardiac rhythm discrimination. In some examples, processing circuitry 40 may store the digitized cardiac EGM in memory 36. Processing circuitry 40 of IMD 14, and/or processing circuitry of another device that retrieves data from IMD 14, may analyze the cardiac EGM.

Communication circuitry 38 may include any suitable hardware, firmware, software or any combination thereof for communicating with another device, such as external device 24, another networked computing device, or another IMD or sensor.

Under the control of processing circuitry 40, communication circuitry 38 may receive downlink telemetry from, as well as send uplink telemetry to external device 24 or another device with the aid of an internal or external antenna, e.g., antenna 26. In addition, processing circuitry 40 may communicate with a networked computing device via an external device (e.g., external device 24 of FIG. 1) and a computer network, such as the Medtronic CareLink® Network. Antenna 26 and communication circuitry 38 may be configured to transmit and/or receive signals via inductive coupling, electromagnetic coupling, Near Field Communication (NFC), Radio Frequency (RF) communication, Bluetooth, Wi-Fi, or other proprietary or non-proprietary wireless communication schemes. Communication antenna 26 may telemeter data at a high frequency, such as around 2.4 gigahertz (GHz).

In some examples, memory 36 includes computer-readable instructions that, when executed by processing circuitry 40, cause IMD 14 and processing circuitry 40 to perform various functions attributed to IMD 14 and processing circuitry 40 herein. Memory 36 may include any volatile, non-volatile, magnetic, optical, or electrical media, such as a random-access memory (RAM), read-only memory (ROM), non-volatile RAM (NVRAM), electrically erasable programmable ROM (EEPROM), flash memory, or any other digital media. Memory 36 may store, as examples, programmed values for one or more operational parameters of IMD 14 and/or data collected by IMD 14, e.g., posture, heart rate, activity level, respiration rate, and other parameters, as well as digitized versions of physiological signals sensed by IMD 14, for transmission to another device using communication circuitry 38.

In the illustrated example, IMD 14 includes processing circuitry 40 and an associated memory 36, sensing circuitry 42, one or more sensors 44, and the communication circuitry 38 coupled to antenna 26 as described above. However, IMD 14 need not include all of these components, or may include additional components.

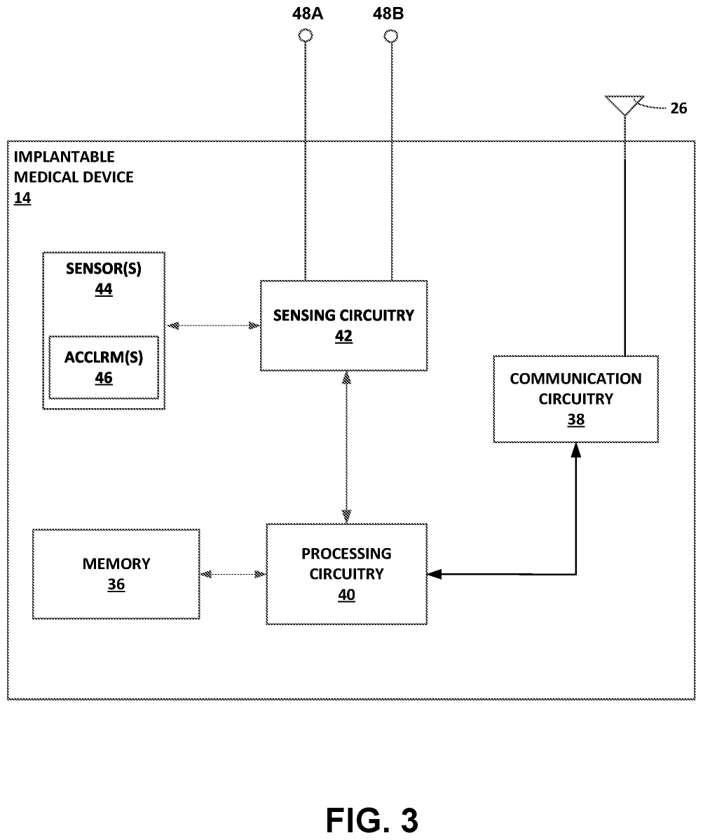

FIG. 4 is a perspective view of an example IMD 14 positioned within a mount 102 including coating masks 104A and 104B (collectively, “masks 104”), according to various examples described in this disclosure. FIG. 5 is a perspective cross-sectional view of a portion of the example IMD 14 positioned within mount 102 of FIG. 4, according to various examples described in this disclosure. Mount 102 is configured to hold IMD 14 during disposition (e.g., during vacuum deposition coating) of biocompatible electrical insulator 16 on housing 20 and to mask electrodes 48 from being coated with biocompatible electrical insulator 16.

Masks 104 are configured to isolate the outer surfaces of electrodes 48 from being coated with biocompatible electrical insulator 16 during disposition of biocompatible electrical insulator 16 on housing 20. For example, masks 104 may be formed with compressible material and to define an electrode cavity 110 configured to receive at least a portion of electrodes 48. In some examples, masks 104 may comprise a polymer, a rubber, a silicone, or any suitably compressible material configured to isolate and/or seal electrode cavity 110 from fluid communication with a volume outside of the cavity, e.g., when masks 104 are compressed to cover 76. In other words, masks 104 may be gaskets configured to mask electrodes 48 during coating of biocompatible electrical insulator 16.

In the example shown, mount 102 includes base 106 and top 108. Top 108 is configured to hold and compress IMD 14 to base 106. In some examples, top 108 and base 106 are configured to compress masks 104 to a surface of housing 20, e.g., to cover 76. In the example shown, top 108 is configured to contact housing 20 with minimal contact area, e.g., at contact point 109. Top 108 may include a structure, e.g., a rigid structure or a compressible structure (not visible in the example shown), configured to extend from a surface of top 108 to contact housing 20, such as a metal bump or post, a rubber bump, post, or the like. In some examples, after disposing and/or coating biocompatible electrical insulator 16 onto at least a portion of housing 20 using mount 102, IMD 14 may be masked and biocompatible electrical insulator 16 may be subsequently disposed and/or coated on housing 20 without mount 102 to cover/coat the contact area. In other areas, the contact area may be substantially small and may be left uncovered/uncoated.

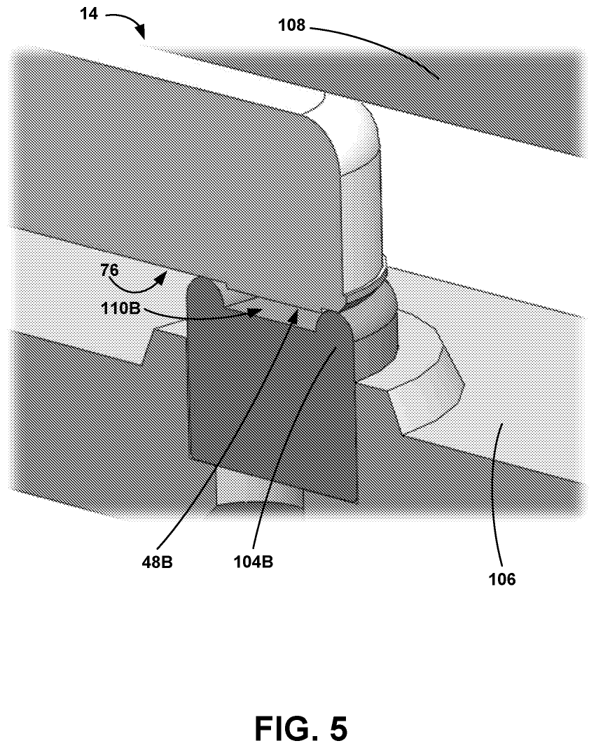

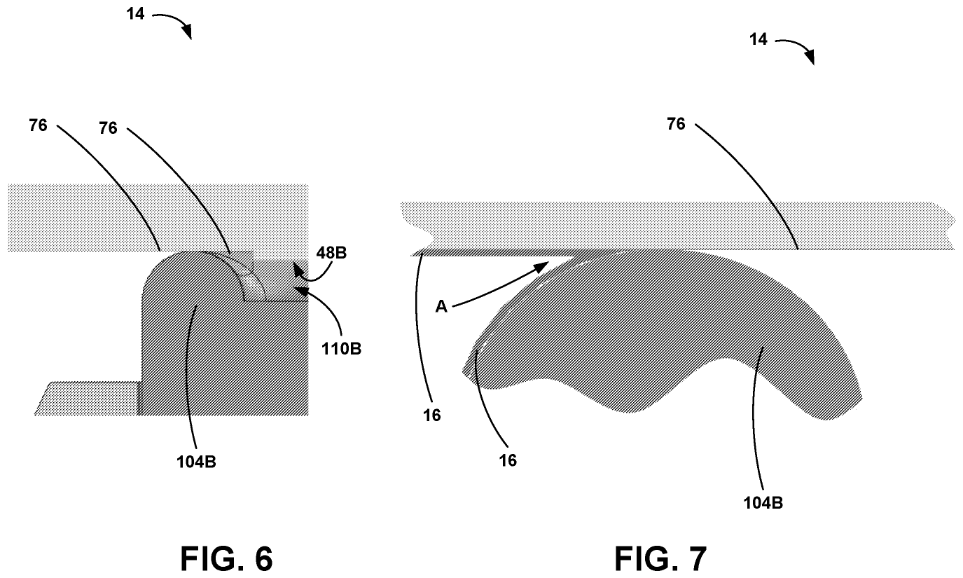

FIG. 6 is an enlarged perspective cross-sectional view of a portion of example IMD 14 and coating mask 104B of FIG. 4, according to various examples described in this disclosure. FIG. 7 is a further enlarged perspective cross-sectional view of the portion of example IMD 14 and coating mask indicated in FIG. 6 including coated biocompatible electrical insulator 16, according to various examples described in this disclosure. FIG. 6 illustrates IMD 14 mounted in mount 102 and mask 104B compressed to cover 76 to seal cavity 110B from a volume outside of cavity 110B with electrode 48B within cavity 110B before disposing and/or coating biocompatible electrical insulator 16. FIG. 7 illustrates a further enlarged view of IMD 14 mounted in mount 102 and mask 104B compressed to cover 76 to seal cavity 110B from a volume outside of cavity 110B with electrode 48B (not shown in FIG. 7) within cavity 110B after disposing and/or coating biocompatible electrical insulator 16.

In the examples shown, electrode 48B is masked from being coating with biocompatible electrical insulator 16, and biocompatible electrical insulator 16 is coated onto portions of cover 76 outside of cavity 110B and onto an outer surface of mask 104B. In some examples, the entire surface of housing, including the outer surfaces of container 15 and cover 76, may be coated with biocompatible electrical insulator 16 except for surfaces within cavity 110B and a corresponding cavity defined by mask 104A (not shown), e.g., and except for surfaces of electrodes 48 (and the contact area from a contact structure of top 108, in some examples).

In some examples, masks 104 are configured to be removed from electrodes 48 without delaminating biocompatible electrical insulator 16 from the surface of housing 20, e.g., masks 104 are configured to be separated from cover 76 without delaminating biocompatible electrical insulator 16 from the outer surface of cover 76. For example, masks 104 may be configured to form a tear portion of the coated biocompatible electrical insulator 16, and the tear portion is configured to tear the coated biocompatible electrical insulator 16 without delaminating the coated biocompatible electrical insulator 16 from the surface of housing 20 upon removal of masks 104 from the electrodes 48 or from the outer surface of cover 76. In some examples, the tear portion comprises an angle, e.g., an acute angle A in the examples shown, between a first portion of biocompatible electrical insulator 16 coated on the surface of housing 20 (e.g., cover 76) and a second portion of biocompatible electrical insulator 16 coated on masks 104, e.g., coated on a surface of masks 104 external to cavity 110B (and the corresponding cavity defined by mask 104A). The vertex of the angle A may define the tear portion between the first and second portions of biocompatible electrical insulator 16.

In the examples shown, after coating biocompatible electrical insulator 16, biocompatible electrical insulator 16 forms angle A at the contact boundary between cover 76 and mask 104B. The vertex of angle A may be a tear portion, e.g., a yield or tear point, of biocompatible electrical insulator 16. That is, upon removal of IMD 14 from mount 102, e.g., separation of cover 76 from masks 104, acute angle A may be a tear portion or weak point of the coated layer of biocompatible electrical insulator 16. In some examples, masks 104 may be configured such that a vertex of angle A defines a boundary along the portions of cover 76 and masks 104 in contact with each other, and the boundary may form a tear portion configured to control where biocompatible electrical insulator 16 separates and/or tears upon separation of masks 104 and cover 76, e.g., along the boundary.

Masks 104 may be configured to form angle A upon coating of biocompatible electrical insulator 16. In some examples, the outer surfaces of masks 104 may be configured to form angle A as an acute angle, e.g., upon compression of the compressible material of masks 104 to housing 20 or cover 76 and coating biocompatible electrical insulator 16. In some examples, masks 104 with an angle of greater than or equal to 5 degrees and less than 90 degrees, or greater than or equal to 10 degrees and less than or equal to 60 degrees, or greater than or equal to 10 degrees and less than or equal to 60 degrees. In some examples, angle A may be controlled and/or varied based on the durometer of masks 104 and the amount of compression of masks 104, e.g., via mount 102.

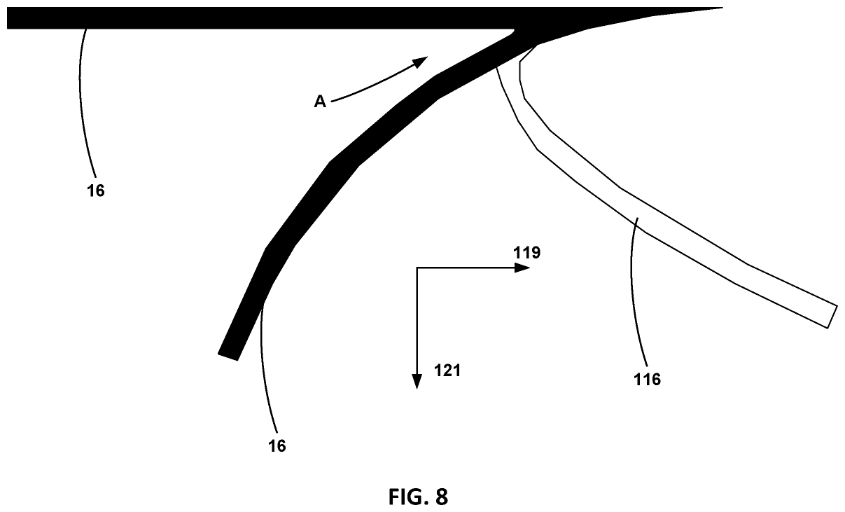

FIG. 8 is a perspective cross-sectional view of biocompatible electrical insulator 16 coated onto IMD 14 of FIG. 7, according to various examples described in this disclosure. FIG. 8 illustrates just biocompatible electrical insulator 16 after coating and before decompression and separation of mask 104 from IMD 14, e.g., with IMD 14 and mask 104 removed from the view. FIG. 8 also illustrates biocompatible electrical insulator 116. In the example shown, biocompatible electrical insulator 116 is biocompatible electrical insulator 16 after coating and decompression of mask 104, but before separation of mask 104 from IMD 14. FIG. 8 illustrates the deformation of biocompatible electrical insulator 16, e.g., resulting in the shape of biocompatible electrical insulator 116, just before tearing of biocompatible electrical insulator 16.

In some examples, biocompatible electrical insulator 16 has a first amount of adhesion to housing 20, e.g., cover 76, and a second amount of adhesion to masks 104 that is greater than the first amount of adhesion. The coated layer of biocompatible electrical insulator 16 may have a cohesion that is greater than its adhesion to housing 20, e.g., container 15 and/or cover 76. In the example shown, IMD 14 is removed from mount 102 by decompressing masks 104, e.g., releasing the compression caused by top 108 of mount 102, and then separating IMD 14 from mount 102. As top 108 reduces the compression of masks 104, the shape of masks 104 may change, and the outer surface of mask 104B at or near housing 20 (e.g., cover 76) may move substantially towards cavity 104B, and the outer surface of mask 104A at or hear housing 20 (e.g., cover 76) may move substantially towards the corresponding cavity defined by mask 104A. In the example shown, the outer surface of mask 104B at or hear housing 20 may move substantially in planar direction 119 and cause a stress at the vertex of angle A, e.g., a bending stress. The bending stress caused by the decompression and movement of masks 104 in planar direction 119 that is substantially parallel with the outer surface of housing 20 (e.g., cover 76) may exceed the yield strength of biocompatible electrical insulator 16 and biocompatible electrical insulator 16 may tear and separate substantially near the vertex of angle A, e.g., along the entire boundary between masks 104 and housing 20 before masks 104 are separated from housing 20 in non-planar direction 121 (e.g., non-planar direction 121 being substantially perpendicular to the outer surface of housing 20 and planar direction 119).

In other examples, the bending stress at the vertex of angle A upon decompression may be less than the yield strength of the coated layer of biocompatible electrical insulator 16 as masks 104 begin to separate from housing 20 in non-planar direction 121. Separation of masks 104 from housing 20 may cause an additional stress on biocompatible electrical insulator 16, e.g., a tensile stress in non-planar direction 121. The bending stress and tensile stress may exceed the yield strength of biocompatible electrical insulator 16, and biocompatible electrical insulator 16 may tear and separate substantially near the vertex of angle A, e.g., along the entire boundary between masks 104 and housing 20 as masks 104 are separated from housing 20 and without delaminating biocompatible electrical insulator 16 from housing 20 (e.g., cover 76). In other words, the bending stress at the vertex of angle A as masks 104 decompress may be enough to tear biocompatible electrical insulator 16 at or near the vertex, or may reduce the tensile stress required to tear biocompatible electrical insulator 16 at or near the vertex. In some examples, the bending stress may reduce the tensile stress required to tear biocompatible electrical insulator 16 at or near the vertex to be less than a tensile stress required peel or separate biocompatible electrical insulator 16 from the outer surface of housing 20, e.g., to be less than the adhesion of biocompatible electrical insulator 16 to housing 20 (e.g., cover 76). In this way, masks 104 may be configured to be removed from IMD 14 and/or electrodes 489 substantially without delaminating biocompatible electrical insulator 16 from housing 20, and in some examples to control the locations of where biocompatible electrical insulator 16 cohesively fails and tears, e.g., the tear portion of biocompatible electrical insulator 16 along a boundary defined by the vertex of angle A after coating biocompatible electrical insulator 16 with masks 104 compressed.

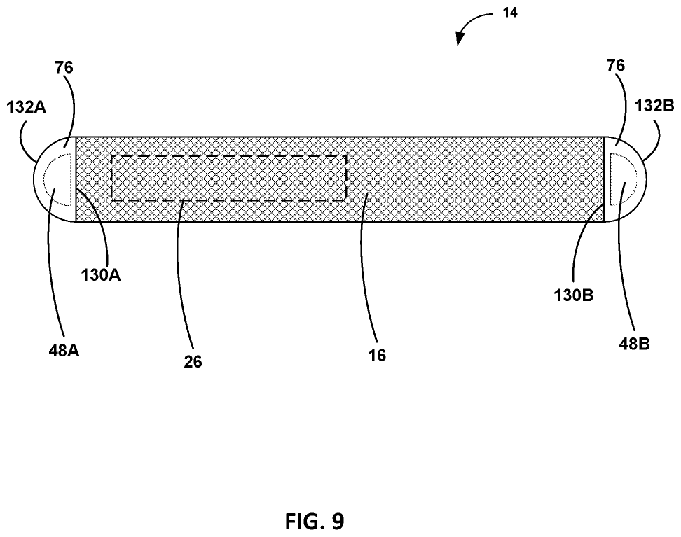

FIG. 9 is front elevation view of IMD 14 including biocompatible electrical insulator 16, according to various examples described in this disclosure. In the example shown, biocompatible electrical insulator 16 is disposed and/or coated on a portion of the outer surface of cover 76, and not coated on a surface of electrodes 48 (and other portions of cover 76, e.g., within cavities 110 and/or contacting masks 104).

In the example shown, biocompatible electrical insulator 16 is disposed on an outer surface of antenna 26 (shown as dashed lines “underneath” biocompatible electrical insulator 16) encapsulating antenna 26 and configured to substantially isolate antenna 26 from tissue and/or fluids of patient 12 upon implantation of IMD 14 within patient 12.

In the example shown, biocompatible electrical insulator 16 includes edges and/or boundaries between coated and uncoated portions of cover 76 at tear portions 130A and 130B. In some examples, biocompatible electrical insulator 16 may be disposed and/or coated on other portions of housing 20, e.g., container 15, and biocompatible electrical insulator 16 may include tear portions along the curved sections of housing 20 in the example shown, e.g., tear portions 132A and 132B. Mask 140A may be configured to define tear portions 130A and 132A and mask 104B may be configured to define tear portions 130B and 132B.

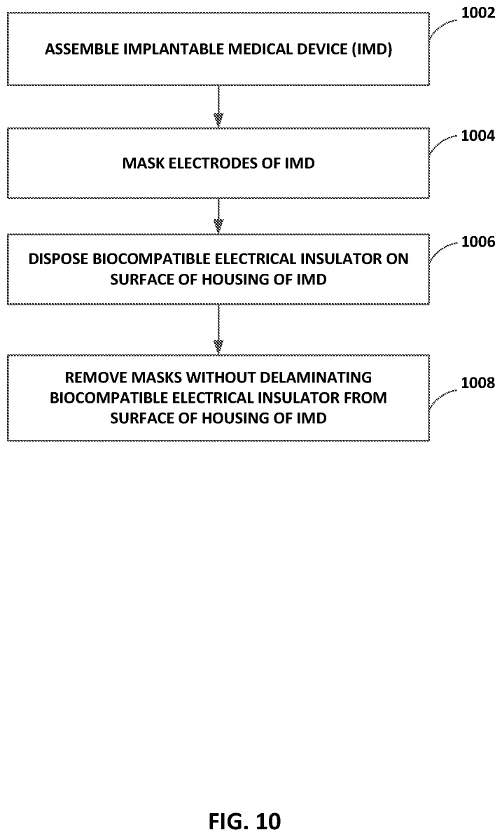

FIG. 10 is a flow diagram of an example method of manufacturing an implantable medical device including an absorbable antibacterial layer, according to various examples described in this disclosure. Although the example technique of FIG. 1 is described with respect to medical systems 10, IMD 14, biocompatible electrical insulator 16, handle 100, and mount 102 of FIGS. 1-9, the example technique of FIG. 10 may be performed using any system including an implantable medical device and a biocompatible electrical insulator described herein. The technique of FIG. 10 may be performed by any suitable user, such as a manufacturer, and the like.

A manufacturer may assemble an IMD including processing circuitry within a housing (1002). For example, a manufacturer may assemble IMD 14 including circuitries 36-42 within housing 20. The manufacturer may mask electrodes 48 prior to disposing biocompatible electrical insulator 16 on housing 20 (1004). For example, the manufacturer may mount IMD 14 within mount 102. For example, the manufacturer may align electrodes 48 with cavities 110 defined by masks 104 and place housing 20 such that electrodes 48 are within cavities 110. The manufacturer may mask electrodes 48 by compressing masks 104 to housing 20 such that masks 104 are compressed to the outer surface of cover 76. In some examples, the manufacturer may compress masks 104 to housing 20 to form angle A between the outer surface of cover 76 and the outer surfaces of masks 104. In some examples, angle A may be an acute angle and/or may be configured to form a tear portion along boundary defined by the vertex of angle A along the perimeters of masks 104 at their outer (e.g., outside of cavities 110) contact areas with cover 76 and after coating biocompatible electrical insulator 16. In some examples, the manufacturer may mask electrodes 48 by compressing masks 104 to seal a cavity, e.g., cavities 110, including electrodes 48 from fluid communication with a volume outside the cavity, e.g., to isolate outer surfaces of the electrodes 48 from biocompatible electrical insulator 16 during coating of biocompatible electrical insulator 16.

The manufacturer may dispose of biocompatible electrical insulator 16 on the housing of IMD 14 such that biocompatible electrical insulator 16 does not interfere with electrical contact between the electrode and tissue and/or fluid of the patient (1006). In some examples, the manufacturer may dispose of biocompatible electrical insulator 16 on the housing of IMD 14 by placing IMD 14 mounted in mount 102 in a vacuum deposition chamber, and coating housing 20 with biocompatible electrical insulator 16 via vacuum deposition.

The manufacturer may remove masks 104 without delaminating biocompatible electrical insulator 16 from the surface of housing 20 (1008). In some examples, the manufacturer may tear biocompatible electrical insulator 16 by separating masks 104 from housing 20. For example, the manufacturer may uncompress and/or decompress masks 104 by releasing IMD 14 from mount 102, and masks 104 may move, flex, uncompress, or decompress to exert a bending stress on the vertex of the angle A along the boundary of masks 104 and housing 20 (e.g., cover 76). The manufacturer may then tear, via releasing IMD 14 from mount 102, biocompatible electrical insulator 16 along the boundary without delaminating the portion of biocompatible electrical insulator 16 coated on housing 20 (e.g., cover 76) from the outer surface of housing 20.

This disclosure includes the following non-limiting examples.

Example 1: An implantable medical device includes a housing configured to house processing circuitry, wherein the processing circuitry is configured to control functioning of the implantable medical device, wherein the housing comprises: an electrically conductive portion defining a cavity configured to receive the processing circuitry; and a dielectric cover configured to cover the cavity and enclose the processing circuitry within the cavity; an electrode positioned on an outer surface of the dielectric cover and connected to the processing circuitry, wherein the processing circuitry is configured to monitor a physiological parameter of a patient via the electrode; and a biocompatible electrical insulator disposed on an outer surface of at least one of the electrically conductive portion or the dielectric cover, wherein the biocompatible electrical insulator is configured to not be disposed on the electrode.

Example 2: The implantable medical device of example 1, further comprising an antenna configured to emit and receive an electromagnetic signal, wherein at least a portion of the antenna is disposed on the outer surface of the dielectric cover, wherein the biocompatible electrical insulator is configured to encapsulate the at least the portion of the antenna disposed on the outer surface of the dielectric cover and to electrically isolate the at least the portion of the antenna from a tissue or a fluid of the patient.

Example 3: The implantable medical device of example 1 or example 2, wherein the processing circuitry is configured to sense electrical signals associated with the electrical activity of a heart of the patient via the electrode.

Example 4: The implantable medical device of any one of examples 1-3, wherein the biocompatible electrical insulator is configured to be disposed over the entire outer surface of the electrically conductive portion of the housing.

Example 5: The implantable medical device of any one of examples 1-4, wherein the biocompatible electrical insulator is configured to be disposed over the entire outer surface of the housing except for the outer surface of the electrode.

Example 6: The implantable medical device of any one of examples 1-4, wherein the biocompatible electrical insulator is parylene.

Example 7: A method of manufacturing the implantable medical device of any one of examples 1 through 6 includes disposing the biocompatible electrical insulator on the housing of the implantable medical device such that the biocompatible electrical insulator does not interfere with electrical contact between the electrode and the tissue or the fluid of the patient.

Example 8: The method of example 7, wherein disposing the biocompatible electrical insulator on the housing of the medical device comprises coating at least a portion of the housing with the biocompatible electrical insulator via vacuum deposition.

Example 9: The method of example 8, further comprising masking the electrode with a mask prior to disposing the biocompatible electrical insulator on the housing.

Example 10: The method of example 9, wherein the mask is configured to be removed from the electrode without delaminating the coated biocompatible electrical insulator from the surface of the housing.

Example 11: The method of example 9 or example 10, wherein the mask is configured to form a tear portion of the coated biocompatible electrical insulator, wherein the tear portion is configured to tear the coated biocompatible electrical insulator without delaminating the coated biocompatible electrical insulator from the surface of the housing upon removal of the mask from the electrode.

Example 12: The method of example 11, wherein the tear portion comprises an acute angle between a first portion of the biocompatible electrical insulator coated on the surface of the housing and a second portion of the biocompatible electrical insulator coated on the mask.

Example 13: The method of example 12, wherein an outer surface of the mask is configured to form the acute angle upon coating the biocompatible electrical insulator.

Example 14: The method of example 13, wherein the mask comprises a compressible material defining a cavity configured to isolate an outer surface of the electrode from the biocompatible electrical insulator during coating of the biocompatible electrical insulator.

Example 15: The method of example 14, wherein the compressible material is configured to be compressed to the housing to seal the cavity from fluid communication with a volume outside the cavity.

Example 16: The method of example 15, wherein an outer surface of the compressible material is configured to form the acute angle upon compression of the compressible material to the housing and coating of the biocompatible electrical insulator.

Example 17: A method includes masking an electrode of an implantable medical device with a mask includes a housing configured to house processing circuitry, wherein the processing circuitry is configured to control functioning of the implantable medical device, wherein the housing comprises: an electrically conductive portion defining a cavity configured to receive the processing circuitry; and a dielectric cover configured to cover the cavity and enclose the processing circuitry within the cavity; and the electrode positioned on an outer surface of the dielectric cover and connected to the processing circuitry, wherein the processing circuitry is configured to monitor a physiological parameter of a patient via the electrode; coating at least a portion of an outer surface of the housing and an outer surface of the mask with a biocompatible electrical insulator; removing the mask without delaminating the coated biocompatible electrical insulator from the surface of the housing.

Example 18: The method of example 17, wherein masking the electrode comprises compressing the compressible material to the housing and about the electrode to seal the cavity from fluid communication with a volume outside the cavity and isolate an outer surface of the electrode from being coated during coating of the biocompatible electrical insulator.

Example 19: The method of example 18, further includes forming, upon compression of the compressible material to the housing, an acute angle between an outer surface of the housing and an outer surface of the compressible material, wherein the acute angle forms tear portion of the biocompatible electrical insulator along a boundary defined by a vertex of the acute angle after coating the biocompatible electrical insulator.

Example 20: The method of example 19, further includes tearing, by separating the mask from the housing, the portion of the biocompatible electrical insulator coated on the housing from the portion of the biocompatible electrical insulator coating on the mask along the boundary without delaminating the portion of the biocompatible electrical insulator coated on the housing from the outer surface of the housing.

The techniques of this disclosure may be implemented in a wide variety of computing devices, medical devices, or any combination thereof. Any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may be implemented, at least in part, in hardware, software, firmware or any combination thereof. For example, various aspects of the techniques may be implemented within one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, or other devices. The terms “processor,” “processor circuitry,” “processing circuitry,” “controller” or “control module” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as random-access memory (RAM), read-only memory (ROM), non-volatile random-access memory (NVRAM), electrically erasable programmable read-only memory (EEPROM), FLASH memory, magnetic media, optical media, or the like that is tangible. The computer-readable storage media may be referred to as non-transitory. A server, client computing device, or any other computing device may also contain a more portable removable memory type to enable easy data transfer or offline data analysis. The instructions may be executed to support one or more aspects of the functionality described in this disclosure.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

Various aspects of this disclosure have been described. These and other aspects are within the scope of the following claims.

1-15. (canceled)

16. An implantable medical device comprising:
a housing configured to house processing circuitry, wherein the processing circuitry is configured to control functioning of the implantable medical device, wherein the housing comprises:
an electrically conductive portion defining a cavity configured to receive the processing circuitry; and
a dielectric cover configured to cover the cavity and enclose the processing circuitry within the cavity;
an electrode positioned on an outer surface of the dielectric cover and connected to the processing circuitry, wherein the processing circuitry is configured to monitor a physiological parameter of a patient via the electrode; and
a biocompatible electrical insulator disposed on an outer surface of the dielectric cover, wherein the biocompatible electrical insulator is configured to not be disposed on the electrode.

17. The implantable medical device of claim 16, further comprising an antenna configured to emit and receive an electromagnetic signal, wherein at least a portion of the antenna is disposed on the outer surface of the dielectric cover, wherein the biocompatible electrical insulator is configured to encapsulate the at least the portion of the antenna disposed on the outer surface of the dielectric cover and to electrically isolate the at least the portion of the antenna from a tissue or a fluid of the patient.

18. The implantable medical device of claim 16, wherein the processing circuitry is configured to sense electrical signals associated with the electrical activity of a heart of the patient via the electrode.

19. The implantable medical device of claim 16, wherein the biocompatible electrical insulator is configured to be disposed over the entire outer surface of the electrically conductive portion of the housing.

20. The implantable medical device of claim 16, wherein the biocompatible electrical insulator is configured to be disposed over the entire outer surface of the housing except for the outer surface of the electrode.

21. The implantable medical device of claim 16, wherein the biocompatible electrical insulator is parylene.

22. A method of manufacturing the implantable medical device of claim 16, the method comprising:
disposing the biocompatible electrical insulator on the dielectric cover of the implantable medical device such that the biocompatible electrical insulator does not interfere with electrical contact between the electrode and the tissue or the fluid of the patient.

23. The method of claim 22, wherein disposing the biocompatible electrical insulator on the dielectric cover of the medical device comprises coating at least a portion of the dielectric cover with the biocompatible electrical insulator via vacuum deposition.

24. The method of claim 23, further comprising masking the electrode with a mask prior to disposing the biocompatible electrical insulator on the dielectric cover.

25. The method of claim 24, wherein the mask is configured to be removed from the electrode without delaminating the coated biocompatible electrical insulator from the surface of the dielectric cover.

26. The method of claim 24, wherein the mask is configured to form a tear portion of the coated biocompatible electrical insulator, wherein the tear portion is configured to tear the coated biocompatible electrical insulator without delaminating the coated biocompatible electrical insulator from the surface of the dielectric cover upon removal of the mask from the electrode.

27. The method of claim 26, wherein the tear portion comprises an acute angle between a first portion of the biocompatible electrical insulator coated on the surface of the dielectric cover and a second portion of the biocompatible electrical insulator coated on the mask.

28. The method of claim 27, wherein an outer surface of the mask is configured to form the acute angle upon coating the biocompatible electrical insulator.

29. The method of claim 28, wherein the mask comprises a compressible material defining a cavity configured to isolate an outer surface of the electrode from the biocompatible electrical insulator during coating of the biocompatible electrical insulator.

30. The method of claim 29, wherein the compressible material is configured to be compressed to the dielectric cover to seal the cavity from fluid communication with a volume outside the cavity.

31. The method of claim 30, wherein an outer surface of the compressible material is configured to form the acute angle upon compression of the compressible material to the housing and coating of the biocompatible electrical insulator.

32. A method comprising:
masking an electrode of an implantable medical device with a mask comprising a compressible material defining a cavity, wherein the implantable medical device comprising:
a housing configured to house processing circuitry, wherein the processing circuitry is configured to control functioning of the implantable medical device, wherein the housing comprises:
an electrically conductive portion defining a cavity configured to receive the processing circuitry; and
a dielectric cover configured to cover the cavity and enclose the processing circuitry within the cavity; and
the electrode positioned on an outer surface of the dielectric cover and connected to the processing circuitry, wherein the processing circuitry is configured to monitor a physiological parameter of a patient via the electrode;
coating at least a portion of an outer surface of the housing and an outer surface of the mask with a biocompatible electrical insulator;
removing the mask without delaminating the coated biocompatible electrical insulator from the surface of the housing.

33. The method of claim 32, wherein masking the electrode comprises compressing the compressible material to the housing and about the electrode to seal the cavity from fluid communication with a volume outside the cavity and isolate an outer surface of the electrode from being coated during coating of the biocompatible electrical insulator.

34. The method of claim 33, further comprising:
forming, upon compression of the compressible material to the housing, an acute angle between an outer surface of the housing and an outer surface of the compressible material, wherein the acute angle forms tear portion of the biocompatible electrical insulator along a boundary defined by a vertex of the acute angle after coating the biocompatible electrical insulator.

35. The method of claim 34, further comprising:
tearing, by separating the mask from the housing, the portion of the biocompatible electrical insulator coated on the housing from the portion of the biocompatible electrical insulator coating on the mask along the boundary without delaminating the portion of the biocompatible electrical insulator coated on the housing from the outer surface of the housing.
